(19) United States
(12) Patent Application Publication
(10) Pub. No.: US 2026/0363578 A1
(43) Pub. Date: Nov. 19, 2026

(54) WEARABLE DEVICE-BASED WALKING METRICS ESTIMATION

(71) Applicant: Google LLC, Mountain View, CA (US)

(72) Inventors: Amir Bahador Farjadian Bejestan, Belmont, MA (US); Alicia Yolanda Kokoszka, San Francisco, CA (US); Jonathan Hsu, Campbell, CA (US); Lawrence Zhengyu Cai, Palo Alto, CA (US); Anupam Pathak, San Carlos, CA (US); Kyle Grant DeHolton, State College, PA (US); Jonathan Wesor Wang, Cupertino, CA (US); Ming-Zher Poh, Belmont, MA (US); Shun Liao, Palo Alto, CA (US)

(21) Appl. No.: 19/676,456

(22) Filed: May 13, 2026

Related U.S. Application Data
(60) Provisional application No. 63/806,529, filed on May 15, 2025.

(51) Int. Cl. A61B 5/11; A61B 5/00; G06N 3/0464

(52) U.S. Cl. CPC A61B 5/112; A61B 5/681; A61B 5/7267; G06N 3/0464; A61B 2562/0219

(57) ABSTRACT

A computing device obtains motion data from at least one sensor and a height parameter associated with a user. The computing device provides the motion data and the height parameter as input to a deep learning model. Using the deep learning model, the computing device estimates a set of walking metrics based on the motion data and the height parameter. The set of walking metrics includes at least one of gait speed, double support time, unilateral step length, swing time, and stance time. This technology enables the estimation of walking metrics using data from a wearable device and a user's height.

### RELATED APPLICATION

This application claims the benefit of U.S. Provisional Application Ser. No. 63/806,529, filed May 15, 2025.

### BACKGROUND

The analysis of gait metrics for more advanced gait metrics such as unilateral swing time or stance time traditionally necessitated the use of specialized laboratory equipment, which is often complex and requires significant space. The advent of inertial measurement units (IMUs) in consumer devices like smartphones and smartwatches offered potential alternatives for gait analysis. However, smartphone-based methods are susceptible to accuracy degradation due to inconsistent device placement, requiring the user to maintain a precise and consistent position for reliable data. While smartwatches provide a more stable on-wrist location and can be used in various scenarios, current smartwatch-based approaches have demonstrated limitations in providing a comprehensive evaluation of a wide range of gait metrics compared to some smartphone implementations.

### SUMMARY

In general, this disclosure describes techniques for estimating walking metrics using data from a wearable device, such as a smartwatch, and a user's height. A computing device on the user's wrist gathers motion data, which is fed into a deep learning model along with the user's height, to figure out things like how fast the user walks and how long the user's feet touch the ground. The techniques described herein enable a wearable device to provide detailed measurements for both the user's left and right feet, offering a more complete picture of the user's walking style without requiring specialized laboratory equipment.

In one example, a method includes obtaining, by a computing device, motion data from at least one sensor, obtaining, by the computing device, a height parameter associated with a user of the computing device, providing, by the computing device, the motion data and the height parameter as input to a deep learning model, and estimating, using the deep learning model, a set of walking metrics based on the motion data and the height parameter, the set of walking metrics including at least one of gait speed, double support time, unilateral step length, swing time, and stance time.

In another example, a computing device includes a processor, at least one sensor configured to generate motion data, and a storage device storing a deep learning model and instructions, wherein when executed by the processor, the instructions cause the computing device to obtain the motion data from the at least one sensor, obtain a height parameter associated with a user, provide the motion data and the height parameter as input to a deep learning model, and receive, from the deep learning model, a set of walking metrics based on the motion data and the height parameter, the set of walking metrics including at least one of gait speed, double support time, unilateral step length, swing time, and stance time.

In another example, a non-transitory computer-readable storage medium includes instructions, that when executed by one or more processors of a computing device, cause the one or more processors to obtain motion data from at least one sensor, obtain a height parameter associated with a user, provide the motion data and the height parameter as input to a deep learning model, and receive, from the deep learning model, a set of walking metrics based on the motion data and the height parameter, the set of walking metrics including at least one of gait speed, double support time, unilateral step length, swing time, and stance time.

The details of one or more examples are set forth in the accompanying drawings and the description below. Other features, objects, and advantages of the disclosure will be apparent from the description and drawings, and from the claims.

### BRIEF DESCRIPTION OF DRAWINGS

FIG. 1 is a block diagram illustrating a system for gait analysis, in accordance with the techniques of this disclosure.

FIG. 2 is a block diagram illustrating components of a wearable computing device, in accordance with the techniques of this disclosure.

FIGS. 3A-3E are conceptual diagrams illustrating aspects of an example machine-learned model trained using differential privacy according to example implementations of the present disclosure.

FIG. 4 is a flow diagram illustrating example operations of a computing device that performs gait analysis, in accordance with the techniques of this disclosure.

### DETAILED DESCRIPTION

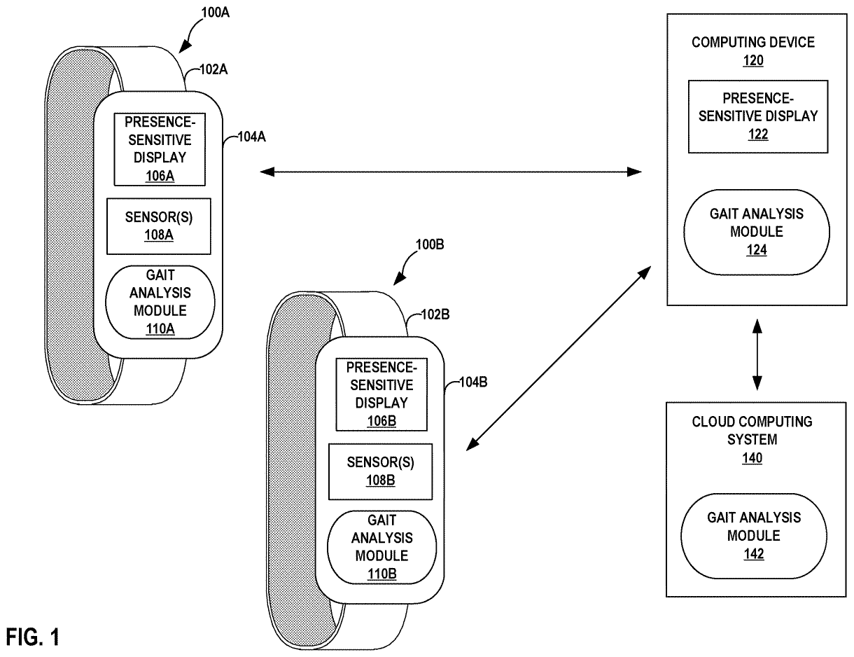

FIG. 1 is a block diagram illustrating a system for gait analysis, in accordance with the techniques of this disclosure. As shown in FIG. 1, the system includes wearable devices 100A and 100B, computing device 120, and cloud computing system 140. Each wearable device, wearable device 100A and wearable device 100B (collectively “wearable devices 100”), includes a respective attachment component 102A and attachment component 102B (collectively “attachment components 102”), a respective housing 104A and 104B (collectively “housings 104”), a respective one of presence-sensitive display 106A and presence-sensitive display 106B (collectively “presence-sensitive displays 106”), a respective one of sensors 108A and sensors 108B (collectively “sensors 108”), and a respective one of gait analysis module 110A and gait analysis module 110B (collectively “gait analysis modules 110”). Computing device 120 includes a presence-sensitive display 122 and a gait analysis module 124. Cloud computing system 140 includes gait analysis module 142.

In the example of FIG. 1, wearable devices 100 are computerized watches. However, in other examples, wearable devices 100 may be a computerized fitness band/tracker, computerized eyewear, computerized headwear, a computerized glove, a computerized ring, etc. In other examples, wearable devices 100 may be any type of mobile computing device that can attach to and be worn on a person's body or clothing.

Attachment components 102 may include a physical portion of a wearable computing device that comes in contact with a body (e.g., tissue, muscle, skin, hair, clothing, etc.) of a user when the user is wearing wearable devices 100 (though, in some examples, portions of housings 104 may also come in contact with the body of the user). For example, in cases where wearable computing device 100A is a watch, attachment component 102A may be a watch band that fits around a user's wrist and comes in contact with the skin of the user. In examples where wearable computing device 100A is eyewear or headwear, attachment component 102A may be a portion of the frame of the eyewear or headwear that fits around a user's head, and when wearable computing device 100A is a glove, attachment component 102A may be the material of the glove that conforms to the fingers and hand of the user. In some examples, wearable computing device 100A can be grasped and held from housing 104A and/or attachment component 102A.

Housings 104 of wearable devices 100 includes a physical portion of a wearable computing device that houses a combination of hardware, software, firmware, and/or other electrical components of wearable device 100. For example, FIG. 1 shows that within housing 104A, wearable computing device 100A may include presence-sensitive display 106, sensors 108A, and gait analysis module 110A. Housing 104 may also include other hardware components and/or software modules not shown in FIG. 1, such as one or more processors, memories, operating systems, applications, and the like.

Presence-sensitive displays 106 may function as an input device and as an output device for wearable devices 100. Presence-sensitive displays 106 may be implemented using various technologies. For instance, presence-sensitive displays 106 may include a presence-sensitive input component, such as a resistive touchscreen, a surface acoustic wave touchscreen, a capacitive touchscreen, a projective capacitance touchscreen, a pressure-sensitive screen, an acoustic pulse recognition touchscreen, or another presence-sensitive display technology. Presence-sensitive displays 106 may function as an output (e.g., display) device using any one or more display components, such as a liquid crystal display (LCD), dot matrix display, light emitting diode (LED) display, microLED, organic light-emitting diode (OLED) display, e-ink, or similar monochrome or color display capable of outputting visible information to a user of wearable devices 100.

Sensors 108 may include various types of inertial measurement units (IMUs), such as accelerometers and gyroscopes. Different types of IMUs, or components thereof, may be used to generate motion data. Some examples of IMUs and their components that may generate motion data include accelerometers, which measure linear acceleration along one or more axes; gyroscopes, which measure angular velocity or rotation rate. Sensors 108 may detect motion and/or orientation of wearable devices 100 and generate motion data based on the detected motion and/or orientation.

Computing device 120 may be a mobile computing device, such as a mobile phone (including a smart phone), a laptop computer, a tablet computer, a wearable computing device, or any other computing device that may operate in conjunction with wearable devices 100. For example, one or more of wearable devices 100 may be communicatively coupled (e.g., via BLUETOOTH, WIFI, etc.) to computing device 120 and operate as a companion device to computing device 120. As shown in FIG. 1, computing device 120 may include presence-sensitive display 122. Presence-sensitive display 122 of computing device 120 may function as an input device for computing device 120 and as an output device and may be similar to presence-sensitive displays 106A and 106B described above with respect to wearable devices 100.

Cloud computing system 140 may include a distributed network of servers, data centers, and software, providing scalable and on-demand computing resources such as processing power, storage, and applications over a network, such as the internet. Cloud computing system 140 may provide various services, including data storage, processing capabilities, and software applications, which computing device 120 and/or wearable devices 100 may utilize to perform functions such as gait analysis or data storage. In some examples, cloud computing system 140 may include a gait analysis module 142 configured to receive data from computing device 120 and/or wearable devices 100 to perform gait analysis remotely.

In accordance with the techniques of this disclosure, a user may wear one or more wearable devices 100, which may be paired or otherwise communicatively coupled to computing device 120. As a user walks, the user's arms move in a back and forth swinging motion. One or more of sensors 108 may detect this swinging motion and generate motion data. More specifically, wearable devices 100A and/or 100B gather motion data from at least one sensor 108A and/or 108B. Prior to collecting or analyzing the motion data, one or more of wearable devices 100 or computing device 120 may output a prompt requesting explicit permission from the user to collect and analyze the motion data for purposes of gait analysis. That is, without explicit permission from the user, wearable devices 100, computing device 120, and cloud computing system 140 may not collect or analyze motion data for purposes of gait analysis.

In various examples, wearable devices 100, computing device 120, and/or cloud computing system 140 may analyze the motion data using one or more gait analysis modules 110, 124, and 142. One or more of gait analysis modules 110, 124, and 142 may determine, using a deep learning model, various metrics of the user's gait, such as step length, gait speed, double support time, unilateral step length, swing time, and stance time. As one example, a user is wearing wearable device 100A. Sensors 108A detect motion of a user's arm and generate motion data based on the detected motion. Wearable device 100A provides the motion data to gait analysis module 110A as well as other data, such as a height of the user (e.g., as a height parameter). Gait analysis module 110A may include a deep learning model that, using the motion data and the height parameter, estimates a set of walking metrics such as gait speed, double support time, and unilateral step length. Beneficially, deep learning model 211 enables wearable computing device 200A to extract a comprehensive suite of both bilateral and unilateral walking metrics (e.g., specific to the left foot and the right foot) using only the motion data collected from a single wrist. This eliminates the need for the user to wear multiple sensors or devices on different limbs to capture foot-specific gait characteristics, thereby reducing user burden while maintaining a high degree of clinical relevance.

Upon estimating the set of walking metrics, gait analysis module 110A may compare the estimated metrics against minimal clinically important difference thresholds to detect meaningful changes in the musculoskeletal or neurological health of the user. For example, gait analysis module 110A may identify a clinically important change in health status if a longitudinal change in the estimated gait speed is between 10 and 20 centimeters per second. Similarly, gait analysis module 110A may identify a clinically important change if a variation in the estimated unilateral step length is 5 centimeters or greater. Such evaluations enable wearable device 100A to autonomously monitor the user for cognitive decline, fall risks, or mobility impairments over time. Furthermore, utilizing wearable device 100A provides a stable and consistent on-wrist placement, avoiding the accuracy degradation and high variability (e.g., a higher standard deviation of absolute error) commonly associated with smartphones that suffer from inconsistent user placement, such as being carried loosely in a hand or a bag. Additionally, wearable device 100A offers phone-less utility, allowing for continuous and seamless gait monitoring in environments where a user might not carry a mobile phone, such as within a home, ensuring that longitudinal health data is captured consistently.

In another example, a user is wearing wearable device 100A on one arm and wearable device 100B on a different arm. Sensors 108A and 108B detect motion of each arm and generate motion data based on the detected motion. In such an example, either wearable device 100A or wearable device 100B may provide the motion data to the other wearable device or may provide the motion data to computing device 120 or cloud computing system 140 for analysis. If, for example, wearable device 100A provides the motion data to wearable device 100B, gait analysis module 110B may use the motion data from both wearable devices 100A and 100B to estimate the set of walking metrics. Similarly, if wearable devices 100 each provide their respective motion data to computing device 120, gait analysis module 124 may use the motion data from both wearable devices 100A and 100B to estimate the set of walking metrics. Similarly, if wearable devices 100 each provide their respective motion data to cloud computing system 140 (either directly or through computing device 120), gait analysis module 142 may use the motion data from both wearable devices 100A and 100B to estimate the set of walking metrics.

While described as a single one of gait analysis modules 110, 124, and 142 performing the walking metrics estimation, in some examples, more than one of gait analysis modules 110, 124, and 142 may perform the walking metrics estimation. For example, each of gait analysis modules 110, 124, and 142 may perform a portion of the walking metrics estimation to distribute computational load across the various devices and systems, which may reduce the amount of stored electrical power consumed by each device while performing the gait analysis, which may increase the battery life of each device.

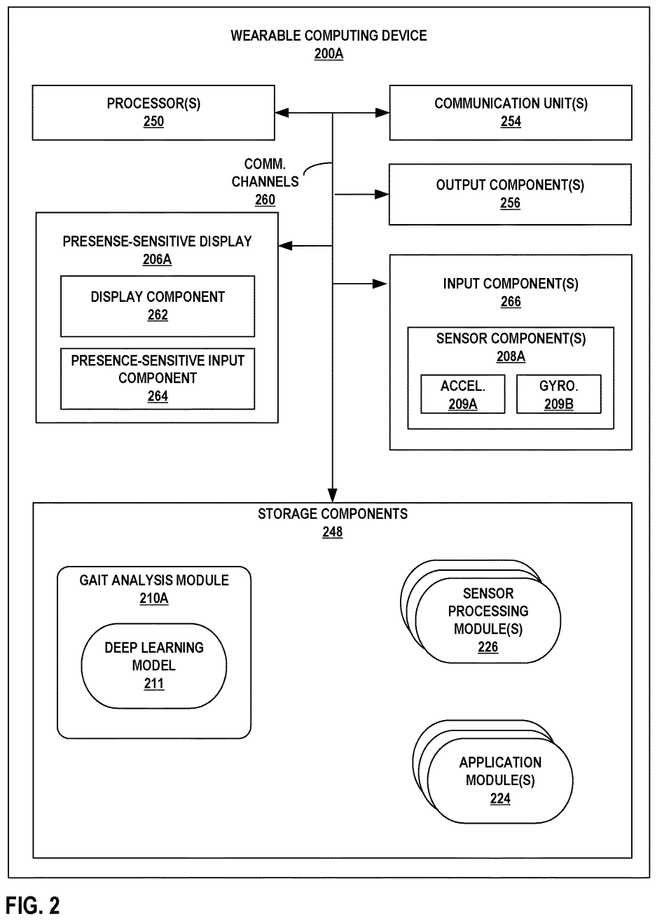

FIG. 2 is a block diagram illustrating components of a wearable computing device configured for gait analysis, in accordance with the techniques of this disclosure. Wearable computing device 200A is one example of wearable device 100A shown in FIG. 1. FIG. 2 illustrates only one particular example of wearable computing device 200A, and many other examples of wearable computing device 200A may be used in other instances and may include a subset of the components included in example wearable computing device 200A or may include additional components not shown in FIG. 2.

As shown in FIG. 2, wearable computing device 200A includes presence-sensitive display 206A, one or more processors 250, one or more input components 266, one or more communication units 254, one or more output components 256, and one or more storage components 248. Presence-sensitive display (PSD) 206A includes display component 262 and presence-sensitive input component 264. Input components 266 include sensor components 208. Storage components 248 of wearable computing device 200A also include gait analysis module 210, deep learning model 211, application modules 224, and sensor processing modules 226.

Communication channels 260 may interconnect each of the components 250, 254, 256, 206A, 266, and 248 for inter-component communications (physically, communicatively, and/or operatively). In some examples, communication channels 260 may include a system bus, a network connection, an inter-process communication data structure, or any other method for communicating data.

One or more input components 266 of wearable computing device 200A may receive input. Examples of input are tactile, audio, and video input. Input components 266 of wearable computing device 200A, in one example, includes a presence-sensitive display, touch-sensitive screen, mouse, keyboard, voice responsive system, video camera, microphone or any other type of device for detecting input from a human or machine.

One or more input components 266 include one or more sensor components 208. Numerous examples of sensor components 208 exist and include any input component configured to obtain motion and/or orientation information about wearable computing device 200A. In some examples, a sensor component may be an input component that obtains physical position, movement, and/or location information of wearable computing device 200A. For instance, sensor components 208 may include one or more multi-axial accelerometers 209A, one or more gyroscopes 209B, and the like. Other sensors may include a magnetometer, a compass sensor, and a step counter sensor, to name a few other non-limiting examples.

One or more output components 256 of wearable computing device 200A may generate output. Examples of output are tactile, audio, and video output. Output components 256 of wearable computing device 200A, in one example, includes a presence-sensitive display, sound card, video graphics adapter card, speaker, liquid crystal display (LCD), or any other type of device for generating output to a human or machine.

One or more communication units 254 of wearable computing device 200A may communicate with external devices via one or more wired and/or wireless networks by transmitting and/or receiving network signals on the one or more networks. Examples of communication units 254 include a BLUETOOTH radio, a WIFI radio, a cellular data radio, other types of wireless network radios, or any other type of device that can send and/or receive information.

Presence-sensitive display (PSD) 206A of wearable computing device 200A includes display component 262 and presence-sensitive input component 264. Display component 262 may be a screen at which information is displayed by PSD 206A and presence-sensitive input component 264 may detect an object at and/or near display component 262. As one example range, presence-sensitive input component 264 may detect an object, such as a finger or stylus that is within two inches or less of display component 262. Presence-sensitive input component 264 may determine a location (e.g., an (x, y) coordinate) of display component 262 at which the object was detected. In another example range, presence-sensitive input component 264 may detect an object six inches or less from display component 262 and other ranges are also possible. Presence-sensitive input component 264 may determine the location of display component 262 selected by a user's finger using capacitive, inductive, and/or optical recognition techniques. In some examples, presence-sensitive input component 264 also provides output to a user using tactile, audio, or video stimuli as described with respect to display component 262. In the example of FIG. 2, PSD 206A presents a user interface.

While illustrated as an internal component of wearable computing device 200A, presence-sensitive display 206A may also represent an external component that shares a data path with wearable computing device 200A for transmitting and/or receiving input and output. For instance, in one example, PSD 206A represents a built-in component of wearable computing device 200A located within and physically connected to the external packaging of wearable computing device 200A (e.g., a screen on a smartwatch). In another example, PSD 206A represents an external component of wearable computing device 200A located outside and physically separated from the packaging of wearable computing device 200A (e.g., a monitor, a projector, etc. that shares a wired and/or wireless data path with a tablet computer).

PSD 206A of wearable computing device 200A may receive tactile input from a user of wearable computing device 200A. PSD 206A may receive indications of the tactile input by detecting one or more gestures from a user of wearable computing device 200A (e.g., the user touching or pointing to one or more locations of PSD 206A with a finger or a stylus pen). PSD 206A may present output to a user. PSD 206A may present the output as a graphical user interface, which may be associated with functionality provided by wearable computing device 200A. For example, PSD 206A may present various user interfaces of components of a computing platform, operating system, applications, or services executing at or accessible by wearable computing device 200A (e.g., an electronic message application, a navigation application, an Internet browser application, a mobile operating system, etc.). A user may interact with a respective user interface to cause wearable computing device 200A to perform operations relating to a function.

PSD 206A of wearable computing device 200A may detect two-dimensional and/or three-dimensional gestures as input from a user of wearable computing device 200A. For instance, a sensor of PSD 206A may detect a user's movement (e.g., moving a hand, an arm, a pen, a stylus, etc.) within a threshold distance of the sensor of PSD 206A. PSD 206A may determine a two-or three-dimensional vector representation of the movement and correlate the vector representation to a gesture input (e.g., a hand-wave, a pinch, a clap, a pen stroke, etc.) that has multiple dimensions. In other words, PSD 206A can detect a multi-dimensional gesture without requiring the user to gesture at or near a screen or surface at which PSD 206A outputs information for display. Instead, PSD 206A can detect a multi-dimensional gesture performed at or near a sensor which may or may not be located near the screen or surface at which PSD 206A outputs information for display.

One or more processors 250 may implement functionality and/or execute instructions within wearable computing device 200A. For example, processors 250 on wearable computing device 200A may receive and execute instructions stored by storage components 248 that execute the functionality of modules 210, 224, and/or 226. The instructions executed by processors 250 may cause wearable computing device 200A to store information within storage components 248 during program execution. Examples of processors 250 include application processors, display controllers, sensor hubs, microcontroller units, and any other hardware configured to function as a processing unit. Processors 250 may execute instructions of modules 210, 224, and/or 226 to cause PSD 206A to render portions of content of display data as one of user interface screen shots at PSD 206A. That is, modules 210, 224, and/or 226 may be operable by processors 250 to perform various actions or functions of wearable computing device 200A.

One or more storage components 248 within wearable computing device 200A may store information for processing during operation of wearable computing device 200A (e.g., wearable computing device 200A may store data accessed by modules 210, 224, and/or 226 during execution at wearable computing device 200A). In some examples, storage component 248 is a temporary memory, meaning that a primary purpose of storage component 248 is not long-term storage. Storage components 248 on wearable computing device 200A may be configured for short-term storage of information as volatile memory and therefore not retain stored contents if powered off. Examples of volatile memories include random access memories (RAM), dynamic random access memories (DRAM), static random access memories (SRAM), and other forms of volatile memories known in the art.

Storage components 248, in some examples, also include one or more computer-readable storage media. Storage components 248 may be configured to store larger amounts of information than volatile memory. Storage components 248 may further be configured for long-term storage of information as non-volatile memory space and retain information after power on/off cycles. Examples of non-volatile memories include magnetic hard discs, optical discs, floppy discs, flash memories, or forms of electrically programmable memories (EPROM) or electrically erasable and programmable (EEPROM) memories. Storage components 248 may store program instructions and/or information (e.g., data) associated with modules 210, 224, and/or 226.

Application modules 224 represent all the various individual applications and services executing at wearable computing device 200A. A user of wearable computing device 200A may interact with an interface (e.g., a graphical user interface) associated with one or more application modules 224 to cause wearable computing device 200A to perform a function. Numerous examples of application modules 224 may exist and include, a fitness application, a calendar application, a personal assistant or prediction engine, a search application, a map or navigation application, a transportation service application (e.g., a bus or train tracking application), a social media application, a game application, an e-mail application, a messaging application, an Internet browser application, or any and all other applications that may execute at wearable computing device 200A. Although shown separately from application modules 224, gait analysis module 210 may be included within one or more of application modules 224 (e.g., included within a fitness application or a health application).

As shown in FIG. 2, wearable computing device 200A may include sensor processing modules 226. In some examples, sensor processing modules 226 may receive outputs from sensor components 208 and generate sensor data that represents the outputs. For instance, each of sensor components 208 may have a corresponding sensor processing module. As an example, a sensor processing module for accelerometer 209A and/or gyroscope 209B may generate motion and/or acceleration values along different axes of a coordinate system in the motion data, where the motion and/or acceleration values are based on hardware outputs of accelerometer component 209A and/or gyroscope component 209B.

Gait analysis module 210 may receive motion data produced by one or more motion sensors. For example, gait analysis module 210 may receive motion data produced by accelerometer 209A and/or gyroscope 209B. In some examples, gait analysis module 210 may receive motion data produced by one or more motion sensors of a wearable computing device communicably coupled to wearable computing device 200A, such as a wearable computing device similar to wearable device 100B shown in FIG. 1.

The motion data produced by one or more motion sensors, such as accelerometer 209A and/or gyroscope 209B, and received by gait analysis module 210 may be in the form of multi-axial accelerometer data, such as tri-axial accelerometer data. For example, the tri-axial accelerometer data may be in the form of three channels of floating point numbers that specify the acceleration measured by the one or more motion sensors along the x, y, and z axes.

The motion data produced by one or more motion sensors and received by gait analysis module 210 may be motion data sensed by the one or more motion sensors over a period of time. For example, the motion data may be motion data sensed by the one or more sensors over a period of approximately 10 seconds. Given a sampling rate of 25 hertz, the motion data may include a total of 256 samples of multi-axial accelerometer data.

Deep learning model 211 of gait analysis module 210 may be any type of machine learning model, including a temporal convolutional network (TCN) model, which may further include a multi-head deep learning model. Deep learning model 211 may be trained to estimate several walking metrics based on motion data and a height parameter associated with a user. In some examples, deep learning model 211 may take accelerometer data (3-axis), gyroscope data (3-axis), and the user's height as input. This sensor data, sampled at 50 Hz, may be divided into 5-second segments with a 1-second overlap for input to deep learning model 211. Deep learning model 211 may include six convolutional layers followed by two dilated convolutional layers and multiple regression heads, one for each walking metric being estimated.

To enhance the robustness and generalizability of deep learning model 211, deep learning model 211 may be trained using data augmentation techniques. These data augmentation techniques may include random three-dimensional rotations, horizontal and vertical flips applied to the training data to simulate variations in how a user might wear wearable computing device 200A. Additionally, random Gaussian noise may be added to the training data to mimic sensor fluctuations. To further enhance the robustness of deep learning model 211, the training data may include motion data collected during various structured walking scenarios. These structured scenarios may comprise a six-minute walk test where a subject walks at a self-paced speed, as well as fast pace walking where the subject walks at a comfortably fast and steady pace. To gather accurate ground truth data for these structured walking scenarios, training subjects may walk along a predefined physical path, such as a 100-foot track. The predefined path may include one or more pressure-sensitive walking mats, such as two 16-foot walking mats positioned along the track, to capture precise reference spatio-temporal walking metrics. The motion data generated by sensor components 208 of wearable computing device 200A during these walking laps is then correlated with the measurements from the walking mats to train deep learning model 211. Including these variations allows deep learning model 211 to accurately estimate metrics across a diverse range of natural walking paces.

The training data may also include simulated asymmetric walking to train deep learning model 211 to recognize unilateral gait abnormalities. Asymmetric walking data may be collected while a subject walks wearing a restrictive device, such as a hinged knee brace. For example, mild asymmetry may be simulated in the training data using a knee brace locked into 60 degrees of flexion and 10 degrees of extension. Furthermore, moderate asymmetry may be simulated using a knee brace locked into 30 degrees of flexion and 10 degrees of extension. Training on such data enables deep learning model 211 to better estimate unilateral swing time and stance time differences.

Deep learning model 211 may be trained using a cross-validation method, dividing subjects into multiple folds for training, validation, and testing, ensuring no overlap in subjects across these splits within each fold. An optimization technique such as the Adam optimizer may be used, and the mean absolute percentage error (MAPE) may serve as the training loss function. The subjects used to generate the training data may be drawn from diverse demographic cohorts to ensure deep learning model 211 generalizes well across different populations and walking styles. For example, the training data may include motion data collected from a first cohort of subjects from a first geographic region, such as the United States, and a second cohort of subjects from a second geographic region, such as Japan. The cohorts may include individuals across a wide range of ages, including older adults, to capture a comprehensive spectrum of natural walking paces, age-related gait variations, and potential gait abnormalities.

Deep learning model 211 may also receive height information for the user in the form of a height parameter. In some examples, the height parameter may be entered by the user via a user interface of wearable computing device 200A. In other examples, the height parameter may be obtained from a data store accessible to wearable computing device 200A.

In addition to the motion data and the height parameter, deep learning model 211 may take one or more additional parameters as input, such as demographic information associated with the user. For example, the one or more additional demographic parameters may comprise age, sex, or weight. That is, when estimating the set of walking metrics, deep learning model 211 may use the motion data, the height parameter, and the one or more additional demographic parameters as input.

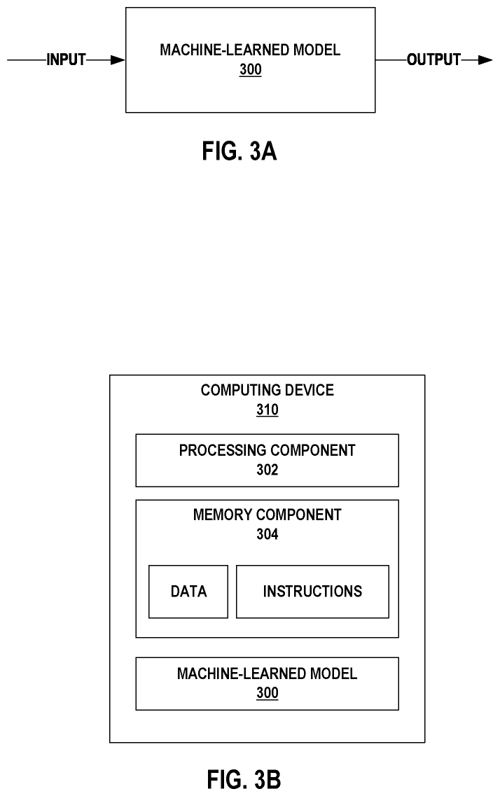

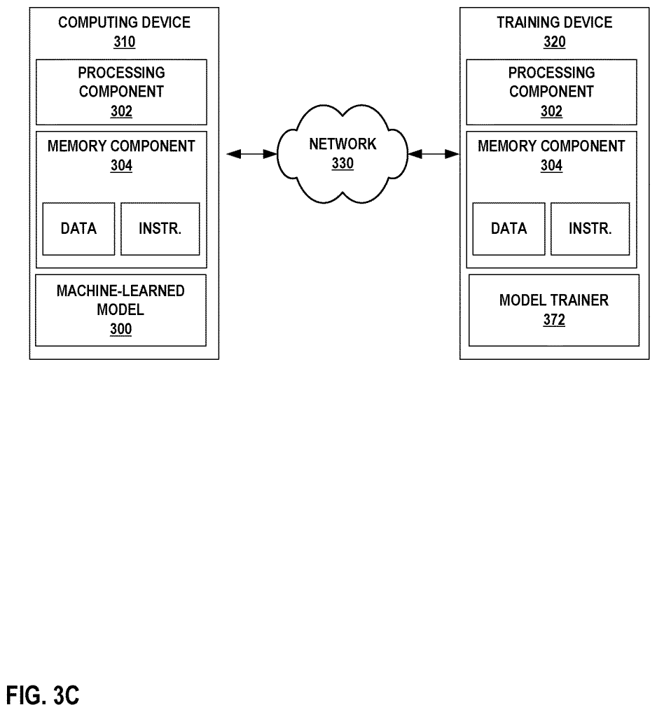

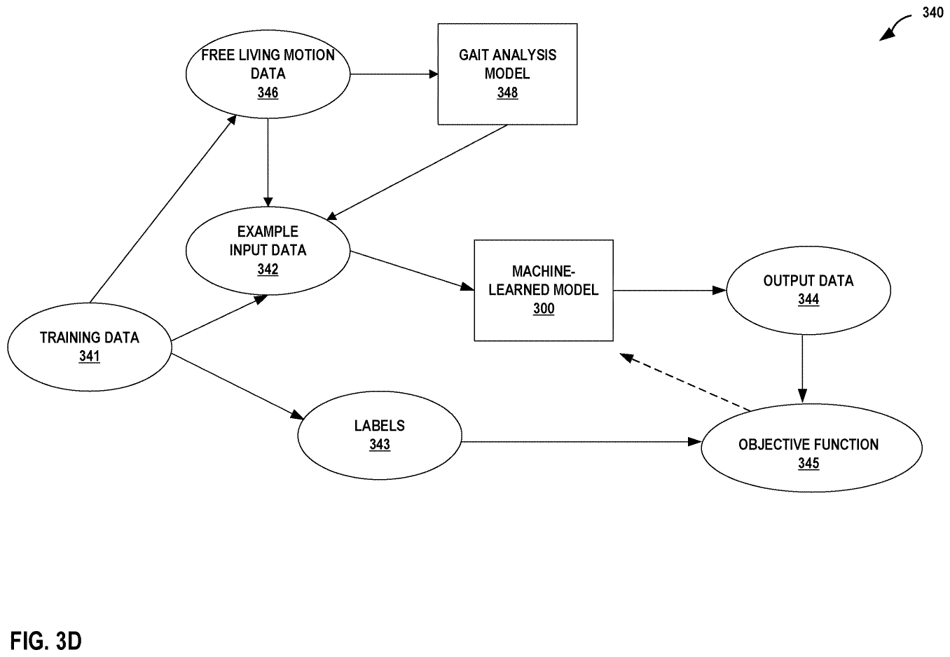

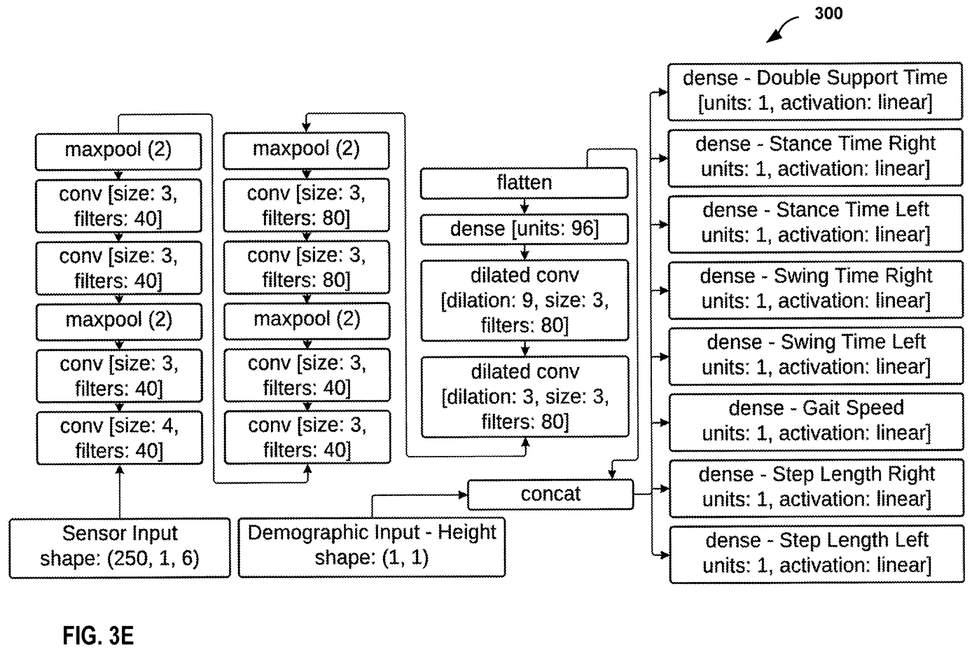

FIGS. 3A-3E are conceptual diagrams illustrating aspects of an example machine-learned model according to example implementations of the present disclosure. FIGS. 3A-3E are described below in the context of gait analysis module 210 and deep learning model 211 of FIG. 2. For example, in some instances, machine-learned model 300, as referenced below, may be an example of deep learning model 211 of FIG. 2.

FIG. 3A depicts a conceptual diagram of an example machine-learned model according to example implementations of the present disclosure. As illustrated in FIG. 3A, in some implementations, machine-learned model 300 is trained to receive input data of one or more types and, in response, provide output data of one or more types. Thus, FIG. 3A illustrates machine-learned model 300 performing inference. For example, the input data received by machine-learned model 300 may be motion data, such as sensor data generated by multi-axial accelerometers, and the output data provided by machine-learned model 300 may be estimated gait metrics for a user that corresponds to the motion data.

The input data may include one or more features that are associated with an instance or an example. In some implementations, the one or more features associated with the instance or example can be organized into a feature vector. In some implementations, the output data can include one or more predictions. Predictions can also be referred to as inferences. Thus, given features associated with a particular instance, machine-learned model 300 can output a prediction for such instance based on the features.

Machine-learned model 300 can be or include one or more of various different types of machine-learned models. In particular, in some implementations, machine-learned model 300 can perform classification, regression, clustering, anomaly detection, recommendation generation, and/or other tasks.

In some implementations, machine-learned model 300 can perform various types of classification based on the input data. For example, machine-learned model 300 can perform binary classification or multiclass classification. In binary classification, the output data can include a classification of the input data into one of two different classes. In multiclass classification, the output data can include a classification of the input data into one (or more) of more than two classes. The classifications can be single label or multi-label. Machine-learned model 300 may perform discrete categorical classification in which the input data is simply classified into one or more classes or categories.

In some implementations, machine-learned model 300 can perform classification in which machine-learned model 300 provides, for each of one or more classes, a numerical value descriptive of a degree to which it is believed that the input data should be classified into the corresponding class. In some instances, the numerical values provided by machine-learned model 300 can be referred to as “confidence scores” that are indicative of a respective confidence associated with classification of the input into the respective class. In some implementations, the confidence scores can be compared to one or more thresholds to render a discrete categorical prediction. In some implementations, only a certain number of classes (e.g., one) with the relatively largest confidence scores can be selected to render a discrete categorical prediction.

Machine-learned model 300 may output a probabilistic classification. For example, machine-learned model 300 may predict, given a sample input, a probability distribution over a set of classes. Thus, rather than outputting only the most likely class to which the sample input should belong, machine-learned model 300 can output, for each class, a probability that the sample input belongs to such class. In some implementations, the probability distribution over all possible classes can sum to one. In some implementations, a Softmax function, or other type of function or layer can be used to squash a set of real values respectively associated with the possible classes to a set of real values in the range (0, 1) that sum to one.

In some examples, the probabilities provided by the probability distribution can be compared to one or more thresholds to render a discrete categorical prediction. In some implementations, only a certain number of classes (e.g., one) with the relatively largest predicted probability can be selected to render a discrete categorical prediction.

In cases in which machine-learned model 300 performs classification, machine-learned model 300 may be trained using supervised learning techniques. For example, machine-learned model 300 may be trained on a training dataset that includes training examples labeled as belonging (or not belonging) to one or more classes. Further details regarding supervised training techniques are provided below in the descriptions of FIGS. 3B through 3E.

In some implementations, machine-learned model 300 can perform regression to provide output data in the form of a continuous numeric value. The continuous numeric value can correspond to any number of different metrics or numeric representations, including, for example, currency values, scores, or other numeric representations. As examples, machine-learned model 300 can perform linear regression, polynomial regression, or nonlinear regression. As examples, machine-learned model 300 can perform simple regression or multiple regression. As described above, in some implementations, a Softmax function or other function or layer can be used to squash a set of real values respectively associated with two or more possible classes to a set of real values in the range (0, 1) that sum to one.

Machine-learned model 300 may, in some cases, act as an agent within an environment. For example, machine-learned model 300 can be trained using reinforcement learning, which will be discussed in further detail below.

In some implementations, machine-learned model 300 can be a parametric model while, in other implementations, machine-learned model 300 can be a non-parametric model. In some implementations, machine-learned model 300 can be a linear model while, in other implementations, machine-learned model 300 can be a non-linear model.

As described above, machine-learned model 300 can be or include one or more of various different types of machine-learned models. Examples of such different types of machine-learned models are provided below for illustration. One or more of the example models described below can be used (e.g., combined) to provide the output data in response to the input data. Additional models beyond the example models provided below can be used as well.

In some implementations, machine-learned model 300 can be or include one or more classifier models such as, for example, linear classification models; quadratic classification models; etc. Machine-learned model 300 may be or include one or more regression models such as, for example, simple linear regression models; multiple linear regression models; logistic regression models; stepwise regression models; multivariate adaptive regression splines; locally estimated scatterplot smoothing models; etc.

In some implementations, machine-learned model 300 can be or include one or more artificial neural networks (also referred to simply as neural networks). A neural network can include a group of connected nodes, which also can be referred to as neurons or perceptrons. A neural network can be organized into one or more layers. Neural networks that include multiple layers can be referred to as “deep” networks. A deep network can include an input layer, an output layer, and one or more hidden layers positioned between the input layer and the output layer. The nodes of the neural network can be connected or non-fully connected.

Machine-learned model 300 can be or include one or more feed forward neural networks. In feed forward networks, the connections between nodes do not form a cycle. For example, each connection can connect a node from an earlier layer to a node from a later layer.

In some instances, machine-learned model 300 can be or include one or more recurrent neural networks. In some instances, at least some of the nodes of a recurrent neural network can form a cycle. Recurrent neural networks can be especially useful for processing input data that is sequential in nature. In particular, in some instances, a recurrent neural network can pass or retain information from a previous portion of the input data sequence to a subsequent portion of the input data sequence through the use of recurrent or directed cyclical node connections.

Example recurrent neural networks include long short-term (LSTM) recurrent neural networks; gated recurrent units; bi-direction recurrent neural networks; continuous time recurrent neural networks; neural history compressors; echo state networks; Elman networks; Jordan networks; recursive neural networks; Hopfield networks; fully recurrent networks; sequence-to-sequence configurations; etc.

In some implementations, machine-learned model 300 can be or include one or more convolutional neural networks. In some instances, a convolutional neural network can include one or more convolutional layers that perform convolutions over input data using learned filters.

Filters can also be referred to as kernels. Convolutional neural networks can be especially useful for vision problems such as when the input data includes imagery such as still images or video. However, convolutional neural networks can also be applied for natural language processing and gait analysis.

In some examples, machine-learned model 300 can be or include one or more generative networks such as, for example, generative adversarial networks. Generative networks can be used to generate new data such as new images or other content.

Machine-learned model 300 may be or include an autoencoder. In some instances, the aim of an autoencoder is to learn a representation (e.g., a lower-dimensional encoding) for a set of data, typically for the purpose of dimensionality reduction. For example, in some instances, an autoencoder can seek to encode the input data and then provide output data that reconstructs the input data from the encoding. Recently, the autoencoder concept has become more widely used for learning generative models of data. In some instances, the autoencoder can include additional losses beyond reconstructing the input data.

Machine-learned model 300 may be or include one or more other forms of artificial neural networks such as, for example, deep Boltzmann machines; deep belief networks; stacked autoencoders; etc. Any of the neural networks described herein can be combined (e.g., stacked) to form more complex networks.

One or more neural networks can be used to provide an embedding based on the input data. For example, the embedding can be a representation of knowledge abstracted from the input data into one or more learned dimensions. In some instances, embeddings can be a useful source for identifying related entities. In some instances, embeddings can be extracted from the output of the network, while in other instances embeddings can be extracted from any hidden node or layer of the network (e.g., a close to final but not final layer of the network). Embeddings can be useful for performing auto suggest next video, product suggestion, entity or object recognition, etc. In some instances, embeddings are useful inputs for downstream models. For example, embeddings can be useful to generalize input data (e.g., search queries) for a downstream model or processing system.

In some implementations, machine-learned model 300 can perform one or more dimensionality reduction techniques such as, for example, principal component analysis; kernel principal component analysis; graph-based kernel principal component analysis; principal component regression; partial least squares regression; Sammon mapping; multidimensional scaling; projection pursuit; linear discriminant analysis; mixture discriminant analysis; quadratic discriminant analysis; generalized discriminant analysis; flexible discriminant analysis; autoencoding; etc.

In some implementations, machine-learned model 300 can perform or be subjected to one or more reinforcement learning techniques such as Markov decision processes; dynamic programming; Q functions or Q-learning; value function approaches; deep Q-networks; differentiable neural computers; asynchronous advantage actor-critics; deterministic policy gradient; etc.

In some implementations, machine-learned model 300 can be an autoregressive model. In some instances, an autoregressive model can specify that the output data depends linearly on its own previous values and on a stochastic term. In some instances, an autoregressive model can take the form of a stochastic difference equation. One example autoregressive model is WaveNet, which is a generative model for raw audio.

In some implementations, machine-learned model 300 can include or form part of a multiple model ensemble. As one example, bootstrap aggregating can be performed, which can also be referred to as “bagging.” In bootstrap aggregating, a training dataset is split into a number of subsets (e.g., through random sampling with replacement) and a plurality of models are respectively trained on the number of subsets. At inference time, respective outputs of the plurality of models can be combined (e.g., through averaging, voting, or other techniques) and used as the output of the ensemble.

One example ensemble is a random forest, which can also be referred to as a random decision forest. Random forests are an ensemble learning method for classification, regression, and other tasks. Random forests are generated by producing a plurality of decision trees at training time. In some instances, at inference time, the class that is the mode of the classes (classification) or the mean prediction (regression) of the individual trees can be used as the output of the forest. Random decision forests can correct for decision trees' tendency to overfit their training set.

Another example ensemble technique is stacking, which can, in some instances, be referred to as stacked generalization. Stacking includes training a combiner model to blend or otherwise combine the predictions of several other machine-learned models. Thus, a plurality of machine-learned models (e.g., of same or different type) can be trained based on training data. In addition, a combiner model can be trained to take the predictions from the other machine-learned models as inputs and, in response, produce a final inference or prediction. In some instances, a single-layer logistic regression model can be used as the combiner model.

Another example ensemble technique is boosting. Boosting can include incrementally building an ensemble by iteratively training weak models and then adding to a final strong model. For example, in some instances, each new model can be trained to emphasize the training examples that previous models misinterpreted (e.g., misclassified). For example, a weight associated with each of such misinterpreted examples can be increased. One common implementation of boosting is AdaBoost, which can also be referred to as Adaptive Boosting. Other example boosting techniques include LPBoost; TotalBoost; BrownBoost; xgboost; MadaBoost, LogitBoost, gradient boosting; etc. Furthermore, any of the models described above (e.g., regression models and artificial neural networks) can be combined to form an ensemble. As an example, an ensemble can include a top level machine-learned model or a heuristic function to combine and/or weight the outputs of the models that form the ensemble.

In some implementations, multiple machine-learned models (e.g., that form an ensemble) can be linked and trained jointly (e.g., through backpropagation of errors sequentially through the model ensemble). However, in some implementations, only a subset (e.g., one) of the jointly trained models is used for inference.

In some implementations, machine-learned model 300 can be used to preprocess the input data for subsequent input into another model. For example, machine-learned model 300 can perform dimensionality reduction techniques and embeddings (e.g., matrix factorization, principal components analysis, singular value decomposition, word2vec/GLOVE, and/or related approaches); clustering; and even classification and regression for downstream consumption. Many of these techniques have been discussed above and will be further discussed below.

As discussed above, machine-learned model 300 can be trained or otherwise configured to receive the input data and, in response, provide the output data. The input data can include different types, forms, or variations of input data. As examples, in various implementations, the input data can include features that describe the content (or portion of content) initially selected by the user, e.g., content of user-selected document or image, links pointing to the user selection, links within the user selection relating to other files available on device or cloud, metadata of user selection, etc. Additionally, with user permission, the input data includes the context of user usage, either obtained from the app itself or from other sources. Examples of usage context include breadth of share (sharing publicly, or with a large group, or privately, or a specific person), context of share, etc. When permitted by the user, additional input data can include the state of the device, e.g., the location of the device, the apps running on the device, etc.

Additionally, with user permission, the input data includes the context of user usage, either obtained from the app itself or from other sources. Examples of usage context include breadth of share (sharing publicly, or with a large group, or privately, or a specific person), context of share, etc. When permitted by the user, additional input data can include the state of the device, e.g., the location of the device, the apps running on the device, etc.

In some implementations, machine-learned model 300 can receive and use the input data in its raw form. In some implementations, the raw input data can be preprocessed. Thus, in addition or alternatively to the raw input data, machine-learned model 300 can receive and use the preprocessed input data.

In some implementations, preprocessing the input data can include extracting one or more additional features from the raw input data. For example, feature extraction techniques can be applied to the input data to generate one or more new, additional features. Example feature extraction techniques include edge detection; corner detection; blob detection; ridge detection; scale-invariant feature transform; motion detection; optical flow; Hough transform; etc.

In some implementations, the extracted features can include or be derived from transformations of the input data into other domains and/or dimensions. As an example, the extracted features can include or be derived from transformations of the input data into the frequency domain. For example, wavelet transformations and/or fast Fourier transforms can be performed on the input data to generate additional features.

In some implementations, the extracted features can include statistics calculated from the input data or certain portions or dimensions of the input data. Example statistics include the mode, mean, maximum, minimum, or other metrics of the input data or portions thereof.

In some implementations, as described above, the input data can be sequential in nature. In some instances, the sequential input data can be generated by sampling or otherwise segmenting a stream of input data. As one example, frames can be extracted from a video. In some implementations, sequential data can be made non-sequential through summarization.

As another example preprocessing technique, portions of the input data can be imputed. For example, additional synthetic input data can be generated through interpolation and/or extrapolation.

As another example preprocessing technique, some or all of the input data can be scaled, standardized, normalized, generalized, and/or regularized. Example regularization techniques include ridge regression; least absolute shrinkage and selection operator (LASSO); elastic net; least-angle regression; cross-validation; L1 regularization; L2 regularization; etc. As one example, some or all of the input data can be normalized by subtracting the mean across a given dimension's feature values from each individual feature value and then dividing by the standard deviation or other metric.

As another example preprocessing technique, some or all or the input data can be quantized or discretized. In some cases, qualitative features or variables included in the input data can be converted to quantitative features or variables. For example, one hot encoding can be performed.

In some examples, dimensionality reduction techniques can be applied to the input data prior to input into machine-learned model 300. Several examples of dimensionality reduction techniques are provided above, including, for example, principal component analysis; kernel principal component analysis; graph-based kernel principal component analysis; principal component regression; partial least squares regression; Sammon mapping; multidimensional scaling; projection pursuit; linear discriminant analysis; mixture discriminant analysis; quadratic discriminant analysis; generalized discriminant analysis; flexible discriminant analysis; autoencoding; etc.

In some implementations, during training, the input data can be intentionally deformed in any number of ways to increase model robustness, generalization, or other qualities. Example techniques to deform the input data include adding noise; changing color, shade, or hue; magnification; segmentation; amplification; etc.

In response to receipt of the input data, machine-learned model 300 can provide the output data. The output data can include different types, forms, or variations of output data. As examples, in various implementations, the output data can include content, either stored locally on the user device or in the cloud, that is relevantly shareable along with the initial content selection.

As discussed above, in some implementations, the output data can include various types of classification data (e.g., binary classification, multiclass classification, single label, multi-label, discrete classification, regressive classification, probabilistic classification, etc.) or can include various types of regressive data (e.g., linear regression, polynomial regression, nonlinear regression, simple regression, multiple regression, etc.). In other instances, the output data can include clustering data, anomaly detection data, recommendation data, or any of the other forms of output data discussed above.

In some implementations, the output data can influence downstream processes or decision making. As one example, in some implementations, the output data can be interpreted and/or acted upon by a rules-based regulator.

The present disclosure provides systems and methods that include or otherwise leverage one or more machine-learned models to estimate a user's gait characteristics, such as step length, gait speed, double support time, swing time, stance time, and asymmetry. Any of the different types or forms of input data described above can be combined with any of the different types or forms of machine-learned models described above to provide any of the different types or forms of output data described above.

The systems and methods of the present disclosure can be implemented by or otherwise executed on one or more computing devices. Example computing devices include user computing devices (e.g., laptops, desktops, and mobile computing devices such as tablets, smartphones, wearable computing devices, etc.); embedded computing devices (e.g., devices embedded within a vehicle, camera, image sensor, industrial machine, satellite, gaming console or controller, or home appliance such as a refrigerator, thermostat, energy meter, home energy manager, smart home assistant, etc.); other computing devices; or combinations thereof.

FIG. 3B illustrates a conceptual diagram of computing device 310, which may be an example of wearable computing device 100A or computing device 120 of FIG. 1. Computing device 310 includes processing component 302, memory component 304 and machine-learned model 300. Computing device 310 may store and implement machine-learned model 300 locally (i.e., on-device). Thus, machine-learned model 300 can be stored at and/or implemented locally by an embedded device or a user computing device such as a mobile device. Output data obtained through local implementation of machine-learned model 300 at the embedded device or the user computing device can be used to improve performance of the embedded device or the user computing device (e.g., an application implemented by the embedded device or the user computing device).

FIG. 3C illustrates a conceptual diagram of an example computing device in communication with an example training computing system that includes a model trainer. FIG. 3C includes computing device 310 communicating with training device 320 over network 330. Computing device 310 may be an example of wearable computing device 100A or computing device 120 of FIG. 1. Machine-learned model 300 described herein can be trained at a training computing system, such as training device 320, and then provided for storage and/or implementation at one or more computing devices, such as computing device 310. For example, model trainer 372 executes locally at training device 320. In some examples, training device 320, including model trainer 372, can be included in or separate from computing device 310 or any other computing device that implements machine-learned model 300.

Computing device 310 that implements machine-learned model 300 or other aspects of the present disclosure and training device 320 that trains machine-learned model 300 can include a number of hardware components that enable performance of the techniques described herein. For example, computing device 310 can include one or more memory devices that store some or all of machine-learned model 300. For example, machine-learned model 300 can be a structured numerical representation that is stored in memory. The one or more memory devices can also include instructions for implementing machine-learned model 300 or performing other operations. Example memory devices include RAM, ROM, EEPROM, EPROM, flash memory devices, magnetic disks, etc., and combinations thereof.

Computing device 310 may also include one or more processing devices that implement some or all of machine-learned model 300 and/or perform other related operations. Example processing devices include one or more of: a central processing unit (CPU); a visual processing unit (VPU); a graphics processing unit (GPU); a tensor processing unit (TPU); a neural processing unit (NPU); a neural processing engine; a core of a CPU, VPU, GPU, TPU, NPU or other processing device; an application specific integrated circuit (ASIC); a field programmable gate array (FPGA); a co-processor; a controller; or combinations of the processing devices described above. Processing devices can be embedded within other hardware components such as, for example, an image sensor, accelerometer, etc.

Training device 320 may perform graph processing techniques or other machine learning techniques using one or more machine learning platforms, frameworks, and/or libraries, such as, for example, TensorFlow, Caffe/Caffe2, Theano, Torch/PyTorch, MXnet, CNTK, etc. In some implementations, machine-learned model 300 may be trained in an offline fashion or an online fashion. In offline training (also known as batch learning), machine-learned model 300 is trained on the entirety of a static set of training data. In online learning, machine-learned model 300 is continuously trained (or re-trained) as new training data becomes available (e.g., while the model is used to perform inference).

Model trainer 372 may perform centralized training of machine-learned model 300 (e.g., based on a centrally stored dataset). In other implementations, decentralized training techniques such as distributed training, federated learning, or the like can be used to train, update, or personalize machine-learned model 300.

Machine-learned model 300 described herein can be trained according to one or more of various different training types or techniques. For example, in some implementations, machine-learned model 300 can be trained by model trainer 372 using supervised learning, in which machine-learned model 300 is trained on a training dataset that includes instances or examples that have labels. The labels can be manually applied by experts, generated through crowd-sourcing, or provided by other techniques (e.g., by physics-based or complex mathematical models). In some implementations, if the user has provided consent, the training examples can be provided by the user computing device. In some implementations, this process can be referred to as personalizing the model.

Once training device 320 has finished training machine-learned model 300, machine-learned model 300 may be installed onto computing device 310. For example, training device 320 may transfer machine-learned model 300 to computing device 310 via network 330, or machine-learned model 300 may be installed in computing device 310 during manufacturing of computing device 310. In some examples, once machine-learned model 300 has been trained at training device 320, training device 320 may perform post-training weight quantization, such as by using TensorFlow Lite libraries, to compress model weights, such as by compressing model weights into 8-bit integers, to enable computing device 310 to perform more efficient on-device inference using machine-learned model 300.

FIG. 3D illustrates a conceptual diagram of training process 340 which is an example training process in which training device 320 may train machine-learned model 300 on training data 341 that includes example input data 342 that has labels 343. Training process 340 is one example training process; other training processes may be used as well.

Training data 341 used by training process 340 can include, upon user permission for use of such data for training, anonymized usage logs of sharing flows, e.g., content items that were shared together, bundled content pieces already identified as belonging together, e.g., from entities in a knowledge graph, etc. In some implementations, training data 341 can include examples of input data 342 that have been assigned labels 343 that correspond to output data 344.

Further, training data 341 may include data from one or both wearable devices 100A and 100B of FIG. 1. To provide consistent evaluation, a five-fold cross-validation method was used when training machine-learned model 300. This method divided test subjects into five groups. Each group includes approximately 160 individuals to train machine-learned model 300, 40 individuals to validation, and 50 individuals to testing. That is, example input data 342, includes the data from the approximately 160 individuals.

No subjects appeared in multiple splits within any single group. A single machine-learned model 300 was trained for each group, resulting in a total of five models. The validation splits were used to tune hyperparameters. The Adam optimizer and the mean absolute percentage error (MAPE) served as the training loss function. Each model's dataset included approximately 226,368 training samples, 57,332 validation samples, and 70,925 testing samples, where each sample comprised a 5-second window.

Each individual example input data in example input data 342 may be motion data over a specified period of time, such as 10 seconds of tri-axial accelerometer data. Example input data 342 may include millions of individual example input data, and the delta value for machine-learned model 300 described above may be the inverse of the number of individual example input data in input data 342.

In some examples, if training data 341 includes example input data 342 that has labels 343, so that each individual example input data of example input data 342 has a label of labels 343. To train machine-learned model 300 to perform gait metric estimation, each individual example input data of example input data 342 may have a label that indicates the portion of the user's gait that corresponds to the individual example input data.

In some examples, training device 320 may incorporate motion data from free living motion data 346 into training data 341 used to train machine-learned model 300. Free living motion data 346 may include motion data generated by motion sensors of computing devices carried by or worn by users as the users go about their everyday activities for multiple consecutive days, such as for three days or more. In general, free living motion data 346 may be unlabeled. As such, training device 320 may determine labels for free living motion data 346 and may include at least a portion of free living motion data 346 and its associated labels in training data 341 used to train machine-learned model 300.

In some examples, training device 320 may determine, based at least in part on the accelerometer magnitudes (i.e., the acceleration forces along the x, y, and z axes) and/or the average accelerometer magnitudes (i.e., the average of the total acceleration forces combining the x, y, and z axes) associated with windows of motion data in free living motion data 346, labels associated with the motion data in free living motion data 346. In particular, training device 320 may determine, based at least in part on the accelerometer magnitudes and/or the average accelerometer magnitudes of motion data in free living motion data 346, whether to label the motion data in free living motion data 346 and whether to select motion data in free living motion data 346 to include in training data 341 used to train machine-learned model 300.

Training device 320 may divide free living motion data 346 into windows of motion data. For example, each window may be approximately 5 seconds of motion data which, when sampled at 100 hertz, includes 512 samples of motion data, such as tri-axial accelerometer data. Training device 320 may determine an average (i.e., mean) accelerometer magnitude for the window, and may for each sample of motion data, subtract the average accelerometer magnitude for the window from the sample of motion data to produce an average accelerometer magnitude with zero mean in the window, such as to remove the effects of gravity. The average accelerometer magnitude of the clipped window may represent the overall score for the window.

In some examples, training device 320 may perform activity recognition of free living motion data 346 using a pre-trained activity recognition model, such as gait analysis model 348, to label free living motion data 346 and to include the labeled free living motion data 346 in training data 341 used to train machine-learned model 300. Gait analysis model 348 may perform activity recognition to determine a probable activity associated with the motion data in the window and to estimate an activity recognition probability for each of the probable activities determined by gait analysis model 348. Gait analysis model 348 may filter out the probable activities having associated activity recognition probabilities that are below specified confidence thresholds, and may label each of the remaining windows of motion data in free living motion data 346 with the associated probable activity having an activity recognition probability that is at or above the confidence threshold, thereby producing labeled motion data that can be incorporated into training data 341 used to train machine-learned model 300.

In some implementations, machine-learned model 300 can be trained by optimizing an objective function, such as objective function 345. For example, in some implementations, objective function 345 may be or include a loss function that compares (e.g., determines a difference between) output data generated by the model from the training data and labels (e.g., ground-truth labels) associated with the training data. For example, the loss function can evaluate a sum or mean of squared differences between the output data and the labels. In some examples, objective function 345 may be or include a cost function that describes a cost of a certain outcome or output data. Other examples of objective function 345 can include margin-based techniques such as, for example, triplet loss or maximum-margin training.

One or more of various optimization techniques can be performed to optimize objective function 345. For example, the optimization technique(s) can minimize or maximize objective function 345. Example optimization techniques include Hessian-based techniques and gradient-based techniques, such as, for example, coordinate descent; gradient descent (e.g., stochastic gradient descent); subgradient methods; etc. Other optimization techniques include black box optimization techniques and heuristics.

In some implementations, backward propagation of errors can be used in conjunction with an optimization technique (e.g., gradient based techniques) to train machine-learned model 300 (e.g., when machine-learned model is a multi-layer model such as an artificial neural network). For example, an iterative cycle of propagation and model parameter (e.g., weights) update can be performed to train machine-learned model 300. Example backpropagation techniques include truncated backpropagation through time, Levenberg-Marquardt backpropagation, etc.

In some implementations, machine-learned model 300 described herein can be trained using unsupervised learning techniques. Unsupervised learning can include inferring a function to describe hidden structure from unlabeled data. For example, a classification or categorization may not be included in the data. Unsupervised learning techniques can be used to produce machine-learned models capable of performing clustering, anomaly detection, learning latent variable models, or other tasks.

Machine-learned model 300 can be trained using semi-supervised techniques which combine aspects of supervised learning and unsupervised learning. Machine-learned model 300 can be trained or otherwise generated through evolutionary techniques or genetic algorithms. In some implementations, machine-learned model 300 described herein can be trained using reinforcement learning. In reinforcement learning, an agent (e.g., model) can take actions in an environment and learn to maximize rewards and/or minimize penalties that result from such actions. Reinforcement learning can differ from the supervised learning problem in that correct input/output pairs are not presented, nor sub-optimal actions explicitly corrected.

In some implementations, one or more generalization techniques can be performed during training to improve the generalization of machine-learned model 300. Generalization techniques can help reduce overfitting of machine-learned model 300 to the training data. Example generalization techniques include dropout techniques; weight decay techniques; batch normalization; early stopping; subset selection; stepwise selection; etc.

In some implementations, machine-learned model 300 described herein can include or otherwise be impacted by a number of hyperparameters, such as, for example, learning rate, number of layers, number of nodes in each layer, number of leaves in a tree, number of clusters; etc. Hyperparameters can affect model performance. Hyperparameters can be hand selected or can be automatically selected through application of techniques such as, for example, grid search; black box optimization techniques (e.g., Bayesian optimization, random search, etc.); gradient-based optimization; etc. Example techniques and/or tools for performing automatic hyperparameter optimization include Hyperopt; Auto-WEKA; Spearmint; Metric Optimization Engine (MOE); etc.

In some implementations, various techniques can be used to optimize and/or adapt the learning rate when the model is trained. Example techniques and/or tools for performing learning rate optimization or adaptation include Adagrad; Adaptive Moment Estimation (ADAM); Adadelta; RMSprop; etc.

In some implementations, transfer learning techniques can be used to provide an initial model from which to begin training of machine-learned model 300 described herein.

In some implementations, machine-learned model 300 described herein can be included in different portions of computer-readable code on a computing device. In one example, machine-learned model 300 can be included in a particular application or program and used (e.g., exclusively) by such a particular application or program. Thus, in one example, a computing device can include a number of applications and one or more of such applications can contain its own respective machine learning library and machine-learned model(s).

In another example, machine-learned model 300 described herein can be included in an operating system of a computing device (e.g., in a central intelligence layer of an operating system) and can be called or otherwise used by one or more applications that interact with the operating system. In some implementations, each application can communicate with the central intelligence layer (and model(s) stored therein) using an application programming interface (API) (e.g., a common, public API across all applications).

In some implementations, the central intelligence layer can communicate with a central device data layer. The central device data layer can be a centralized repository of data for the computing device. The central device data layer can communicate with a number of other components of the computing device, such as, for example, one or more sensors, a context manager, a device state component, and/or additional components. In some implementations, the central device data layer can communicate with each device component using an API (e.g., a private API).

The technology discussed herein makes reference to servers, databases, software applications, and other computer-based systems, as well as actions taken and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

In addition, the machine learning techniques described herein are readily interchangeable and combinable. Although certain example techniques have been described, many others exist and can be used in conjunction with aspects of the present disclosure.

Further to the descriptions above, a user may be provided with controls allowing the user to make an election as to both if and when systems, programs or features described herein may enable collection of user information (e.g., information about a user's social network, social actions or activities, profession, a user's preferences, or a user's current location), and if the user is sent content or communications from a server. In addition, certain data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over what information is collected about the user, how that information is used, and what information is provided to the user.

FIG. 3E illustrates a conceptual diagram illustrating an example machine-learned model architecture. As shown in FIG. 3E, machine-learned model 300 may be a convolutional neural network (CNN) that includes a CNN extractor, a dilated temporal convolutional network (TCN), and regression heads. The input to machine-learned model 300 may be 6-axis inertial measurement unit (IMU) data, a user height parameter, as well as other demographic information for the user.

As shown in FIG. 3E, the CNN feature extractor receives 250-timestep sequence of six sensor readings as input. Initially, the sensor data is processed by the CNN feature extractor that includes four blocks where each block includes two convolutional layers (conv) followed by a max pooling layer (maxpool). Each convolutional layer employs a kernel size of 3 and extracts 40 or 80 feature maps from the input signal, while the subsequent max pooling layers reduce spatial dimensionality via a pool size of 2. The output of the CNN feature extractor is passed to a dilated temporal convolutional network (TCN). The TCN includes two dilated convolutional layers followed by a dense layer with 96 units and then a flatten layer that flattens the output into a one-dimensional vector (dilated conv). These dilated convolutional layers utilize kernel sizes of 3, filter counts of 80, and dilation rates of 3 and 9 respectively, enabling the network to efficiently capture long-range temporal dependencies within the gait cycle without a prohibitive increase in computational complexity. In some implementations, the temporal convolutional network of deep learning model 211 comprises approximately 130,000 parameters. This compact model size allows wearable computing device 200A to efficiently execute deep learning model 211 locally using one or more processors 250, conserving battery power stored in wearable computing device 200A while providing rapid estimation of the walking metrics directly on the device. The combined feature vector is processed by a series of eight fully connected regression heads, each comprising a dense layer with a single unit and a linear activation function to predict continuous gait parameters including double support time, stance time (right & left), swing time (right & left), gait speed, and step length (right & left). These continuous gait parameters output by the regression heads of deep learning model 211 represent specific spatio-temporal measurements of the user's movements. For instance, the gait speed represents a distance an individual travels divided by a time taken, typically measured in centimeters per second. The double support time represents a proportion or percentage of a gait cycle when both feet of the user are simultaneously on the ground. The unilateral step length represents a distance from an initial contact of one foot to an initial contact of the other foot, typically measured in centimeters. The unilateral swing time represents a duration within the gait cycle when the foot is not in contact with the ground, typically measured in milliseconds. The unilateral stance time represents a duration within the gait cycle when the foot is in contact with the ground, also typically measured in milliseconds. This architecture allows for automated and accurate prediction of key gait characteristics from sensor data and demographic information.

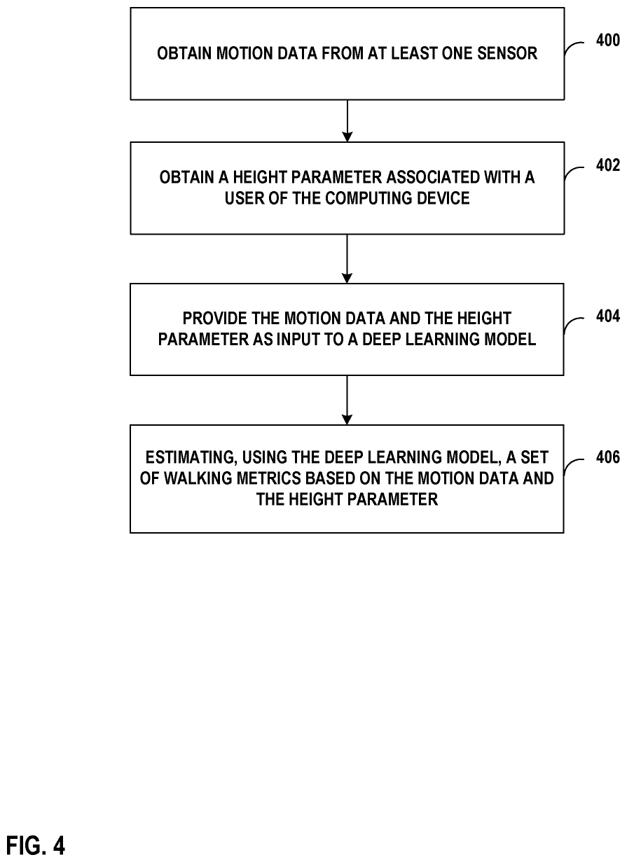

FIG. 4 is a flow diagram illustrating example operations of a computing device that performs gait analysis, in accordance with the techniques of this disclosure. The techniques of FIG. 4 may be performed by one or more processors of a computing device, such as wearable devices 100, computing device 120, and/or cloud computing system 140 of FIG. 1. For purposes of illustration only, the techniques of FIG. 4 are described within the context of wearable computing device 100A of FIG. 1, although computing devices having configurations different than that of computing device 100A, may perform the techniques of FIG. 4.

In accordance with one or more aspects of this disclosure, wearable computing device 100A may obtain motion data from at least one sensor 108A (400). For example, sensor 108A may detect motion of a user's arm and generate motion data based on the detected motion. Wearable computing device 100A may obtain a height parameter associated with the user (402). For example, wearable computing device 100A may request height information from the user or obtain the height information from a data store accessible to wearable computing device 100A. Wearable computing device 100A may provide the motion data and the height parameter as input to a deep learning model (404). Wearable computing device 100A may estimate, using the deep learning model, a set of walking metrics based on the motion data and the height parameter (406). The set of walking metrics may include at least one of gait speed, double support time, and unilateral step length.

In one or more examples, the functions described may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media, which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Disk and disc, as used, includes compact disc (CD), optical disc, digital versatile disc (DVD), Blu-ray disc, where disks usually reproduce data magnetically, while discs reproduce data optically with lasers. Combinations of the above should also be included within the scope of computer-readable media.

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used may refer to any of the foregoing structures or any other structure suitable for implementation of the techniques described. In addition, in some aspects, the functionality described may be provided within dedicated hardware and/or software modules. Also, the techniques could be fully implemented in one or more circuits or logic elements.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

It is to be recognized that depending on the example, certain acts or events of any of the methods described herein can be performed in a different sequence, may be added, merged, or left out altogether (e.g., not all described acts or events are necessary for the practice of the method). Moreover, in certain embodiments, acts or events may be performed concurrently, e.g., through multi-threaded processing, interrupt processing, or multiple processors, rather than sequentially.

In some examples, a computer-readable storage medium includes a non-transitory medium. In some examples, the term “non-transitory” indicates that the storage medium is not embodied in a carrier wave or a propagated signal. In certain examples, a non-transitory storage medium may store data that can, over time, change (e.g., in RAM or cache). Although certain examples are described as outputting various information for display, techniques of the disclosure may output such information in other forms, such as audio, holographical, or haptic forms, to name only a few examples, in accordance with techniques of the disclosure.

### EXAMPLES

Example 1. A method comprising obtaining, by a computing device, motion data from at least one sensor, obtaining, by the computing device, a height parameter associated with a user of the computing device, providing, by the computing device, the motion data and the height parameter as input to a deep learning model, and estimating, using the deep learning model, a set of walking metrics based on the motion data and the height parameter, the set of walking metrics including at least one of gait speed, double support time, unilateral step length, swing time, and stance time.

Example 2. The method of example 1, wherein the deep learning model comprises a temporal convolutional network model.

Example 3. The method of example 2, wherein the temporal convolutional network model comprises a multi-head deep learning model.

Example 4. The method of any of examples 1-3, wherein the at least one sensor includes an accelerometer and a gyroscope, and wherein obtaining the motion data comprises obtaining accelerometer data from the accelerometer and gyroscope data from the gyroscope.

Example 5. The method of example 4, further comprising dividing the accelerometer data and the gyroscope data into a plurality of segments, wherein each segment comprises data collected over a duration of approximately five seconds with an overlap of approximately 1 second between consecutive segments.

Example 6. The method of any of examples 1-5, wherein estimating the set of walking metrics further comprises estimating unilateral step length for a left foot and a right foot.

Example 7. The method of any of examples 1-6, further comprising training the deep learning model using data augmentation, the data augmentation comprising at least one of random three-dimensional rotations, horizontal flips, vertical flips, and random Gaussian noise applied to training data.

Example 8. The method of any of examples 1-7, wherein estimating the set of walking metrics comprises estimating the set of walking metrics using the deep learning model executing on a processor of the computing device.

Example 9. The method of any of examples 1-8, wherein obtaining the height parameter comprises obtaining the height parameter via a user interface of the computing device.

Example 10. The method of any of examples 1-9, wherein obtaining the height parameter comprises obtaining the height parameter from a data store accessible by the computing device.

Example 11. The method of any of examples 1-10, wherein the deep learning model is configured to receive accelerometer data, gyroscope data, and the height parameter as input.

Example 12. The method of any of examples 1-11, further comprising obtaining, by the computing device, one or more additional demographic parameters associated with the user and providing the one or more additional demographic parameters as input to the deep learning model, wherein estimating the set of walking metrics is further based on the one or more additional demographic parameters.

Example 13. A computing device comprising one or more processors, at least one sensor configured to generate inertial measurement unit data, and a storage device storing a deep learning model and instructions, wherein when executed by the one or more processors, the instructions cause the computing device to obtain the inertial measurement unit data from the at least one sensor, obtain a height parameter associated with a user, provide the inertial measurement unit data and the height parameter as input to the deep learning model, and receive, from the deep learning model, a set of walking metrics based on the inertial measurement unit data and the height parameter, the set of walking metrics including at least one of gait speed, double support time, unilateral step length, swing time, and stance time.

Example 14. The computing device of example 13, wherein the deep learning model comprises a temporal convolutional network model.

Example 15. The computing device of example 14, wherein the temporal convolutional network model comprises a multi-head deep learning model.

Example 16. The computing device of any of examples 13-15, wherein the at least one sensor includes an accelerometer and a gyroscope, and wherein the inertial measurement unit data comprises accelerometer data obtained from the accelerometer and gyroscope data obtained from the gyroscope.

Example 17. The computing device of example 16, wherein the instructions further cause the one or more processors to divide the accelerometer data and the gyroscope data into a plurality of segments, wherein each segment comprises data collected over a duration of approximately five seconds with an overlap of approximately 1 second between consecutive segments.

Example 18. The computing device of any of examples 13-17, wherein the set of walking metrics further comprises unilateral step length for a left foot and a right foot.

Example 19. The computing device of any of examples 13-18, wherein the deep learning model is trained using data augmentation, the data augmentation comprising at least one of random three-dimensional rotations, horizontal flips, vertical flips, and random Gaussian noise applied to training data.

Example 20. The computing device of any of examples 13-19, wherein estimating the set of walking metrics comprises estimating the set of walking metrics using the deep learning model executing on the one or more processors.

Example 21. The computing device of any of examples 13-20, further comprising a display device, wherein the instructions further cause the one or more processors to output, for display by the display device, a user interface, and obtain the height parameter via the user interface.

Example 22. The computing device of any of examples 13-21, further comprising a data store, wherein the instructions further cause the one or more processors to obtain the height parameter from the data store.

Example 23. The computing device of any of examples 13-22, wherein the deep learning model is configured to receive accelerometer data, gyroscope data, and the height parameter as input.

Example 24. The computing device of any of examples 13-23, wherein the instructions further cause the one or more processors to obtain one or more additional demographic parameters associated with the user, and provide the one or more additional demographic parameters as input to the deep learning model, wherein estimating the set of walking metrics is further based on the one or more additional demographic parameters.

Example 25. A non-transitory computer-readable storage medium comprising instructions, that when executed by one or more processors of a computing device, cause the one or more processors to obtain inertial measurement unit data from at least one sensor, obtain a height parameter associated with a user, provide the inertial measurement unit data and the height parameter as input to a deep learning model, and receive, from the deep learning model, a set of walking metrics based on the inertial measurement unit data and the height parameter, the set of walking metrics including at least one of gait speed, double support time, unilateral step length, swing time, and stance time.

Example 26. The non-transitory computer-readable storage medium of example 25, wherein the deep learning model comprises a temporal convolutional network model.

Example 27. The non-transitory computer-readable storage medium of example 26, wherein the temporal convolutional network model comprises a multi-head deep learning model.

Example 28. The non-transitory computer-readable storage medium of any of examples 25-27, wherein the at least one sensor includes an accelerometer and a gyroscope, and wherein obtaining the inertial measurement unit data comprises obtaining accelerometer data from the accelerometer and gyroscope data from the gyroscope.

Example 29. The non-transitory computer-readable storage medium of example 28, wherein the instructions further cause the one or more processors to divide the accelerometer data and the gyroscope data into a plurality of segments, wherein each segment comprises data collected over a duration of approximately five seconds with an overlap of approximately 1 second between consecutive segments.

Example 30. The non-transitory computer-readable storage medium of any of examples 25-29, wherein the instructions further cause the one or more processors to estimate the set of walking metrics by at least causing the one or more processors to estimate unilateral step length for a left foot and a right foot.

Example 31. The non-transitory computer-readable storage medium of any of examples 25-30, wherein the deep learning model is trained using data augmentation, the data augmentation comprising at least one of random three-dimensional rotations, horizontal flips, vertical flips, and random Gaussian noise applied to training data.

Example 32. The non-transitory computer-readable storage medium of any of examples 25-31, wherein the instructions cause the one or more processors to estimate the set of walking metrics using the deep learning model executing on the one or more processors of the computing device.

Example 33. The non-transitory computer-readable storage medium of any of examples 25-32, wherein the instructions further cause the one or more processors to obtain the height parameter by at least causing the one or more processors to obtain the height parameter via a user interface of the computing device.

Example 34. The non-transitory computer-readable storage medium of any of examples 25-33, wherein the instructions further cause the one or more processors to obtain the height parameter by at least causing the one or more processors to obtain the height parameter from a data store accessible by the computing device.

Example 35. The non-transitory computer-readable storage medium of any of examples 25-34, wherein the deep learning model is configured to receive accelerometer data, gyroscope data, and the height parameter as input.

Example 36. The non-transitory computer-readable storage medium of any of examples 25-35, wherein the instructions further cause the one or more processors to obtain one or more additional demographic parameters associated with the user, and provide the one or more additional demographic parameters as input to the deep learning model, wherein estimating the set of walking metrics is further based on the one or more additional demographic parameters.

Example 37. A computing device comprising means for performing any combination of examples 1-12.

Example 38. A computer program product comprising one or more instructions that, when executed by a computing device, cause the computing device to perform any combination of examples 1-12.

Various examples have been described. These and other examples are within the scope of the following claims.

What is claimed is:

1. A method comprising:
obtaining, by a computing device, motion data from at least one sensor;
obtaining, by the computing device, a height parameter associated with a user of the computing device;
providing, by the computing device, the motion data and the height parameter as input to a deep learning model; and
estimating, using the deep learning model, a set of walking metrics based on the motion data and the height parameter, the set of walking metrics including at least one of gait speed, double support time, unilateral step length, swing time, and stance time.

2. The method of claim 1, wherein the deep learning model comprises a temporal convolutional network model.

3. The method of claim 2, wherein the temporal convolutional network model comprises a multi-head deep learning model.

4. The method of claim 1, wherein the at least one sensor includes an accelerometer and a gyroscope, and wherein obtaining the motion data comprises obtaining accelerometer data from the accelerometer and gyroscope data from the gyroscope.

5. The method of claim 4, further comprising:
dividing the accelerometer data and the gyroscope data into a plurality of segments, wherein each segment comprises data collected over a duration of approximately five seconds with an overlap of approximately 1 second between consecutive segments.

6. The method of claim 1, wherein estimating the set of walking metrics further comprises estimating unilateral step length for a left foot and a right foot.

7. The method of claim 1, further comprising training the deep learning model using data augmentation, the data augmentation comprising at least one of random three-dimensional rotations, horizontal flips, vertical flips, and random Gaussian noise applied to training data.

8. The method of claim 1, wherein estimating the set of walking metrics comprises estimating the set of walking metrics using the deep learning model executing on a processor of the computing device.

9. The method of claim 1, wherein obtaining the height parameter comprises obtaining the height parameter via a user interface of the computing device.

10. The method of claim 1, wherein obtaining the height parameter comprises obtaining the height parameter from a data store accessible by the computing device.

11. The method of claim 1, wherein the deep learning model is configured to receive accelerometer data, gyroscope data, and the height parameter as input.

12. The method of claim 1, further comprising obtaining, by the computing device, one or more additional demographic parameters associated with the user and providing the one or more additional demographic parameters as input to the deep learning model, wherein estimating the set of walking metrics is further based on the one or more additional demographic parameters.

13. A computing device comprising:
one or more processors;
at least one sensor configured to generate inertial measurement unit data; and
a storage device storing a deep learning model and instructions, wherein when executed by the one or more processors, the instructions cause the computing device to:
obtain the inertial measurement unit data from the at least one sensor;
obtain a height parameter associated with a user;
provide the inertial measurement unit data and the height parameter as input to the deep learning model; and
receive, from the deep learning model, a set of walking metrics based on the inertial measurement unit data and the height parameter, the set of walking metrics including at least one of gait speed, double support time, unilateral step length, swing time, and stance time.

14. The computing device of claim 13, wherein the deep learning model comprises a temporal convolutional network model, and wherein the temporal convolutional network model comprises a multi-head deep learning model.

15. The computing device of claim 13, wherein the at least one sensor includes an accelerometer and a gyroscope, and wherein the inertial measurement unit data comprises accelerometer data obtained from the accelerometer and gyroscope data obtained from the gyroscope.

16. The computing device of claim 15, wherein the instructions further cause the one or more processors to:
divide the accelerometer data and the gyroscope data into a plurality of segments, wherein each segment comprises data collected over a duration of approximately five seconds with an overlap of approximately 1 second between consecutive segments.

17. The computing device of claim 13, wherein the instructions further cause the one or more processors to:
obtain one or more additional demographic parameters associated with the user; and
provide the one or more additional demographic parameters as input to the deep learning model,
wherein the set of walking metrics is further based on the one or more additional demographic parameters.

18. A non-transitory computer-readable storage medium comprising instructions, that when executed by one or more processors of a computing device, cause the one or more processors to:
obtain inertial measurement unit data from at least one sensor;
obtain a height parameter associated with a user;
provide the inertial measurement unit data and the height parameter as input to a deep learning model; and
receive, from the deep learning model, a set of walking metrics based on the inertial measurement unit data and the height parameter, the set of walking metrics including at least one of gait speed, double support time, unilateral step length, swing time, and stance time.

19. The non-transitory computer-readable storage medium of claim 18,
wherein the at least one sensor includes an accelerometer and a gyroscope;
wherein obtaining the inertial measurement unit data comprises obtaining accelerometer data from the accelerometer and gyroscope data from the gyroscope; and
wherein the instructions further cause the one or more processors to:
divide the accelerometer data and the gyroscope data into a plurality of segments, wherein each segment comprises data collected over a duration of approximately five seconds with an overlap of approximately 1 second between consecutive segments.

20. The non-transitory computer-readable storage medium of claim 18, wherein the instructions further cause the one or more processors to:
obtain one or more additional demographic parameters associated with the user; and
provide the one or more additional demographic parameters as input to the deep learning model,
wherein the set of walking metrics is further based on the one or more additional demographic parameters.
